(19) United States
(12) Patent Application Publication
(10) Pub. No.: US 2011/0106234 A1
(43) Pub. Date: May 5, 2011

(54) INTERLUMINAL MEDICAL TREATMENT DEVICES AND METHODS

(76) Inventor: Axel Grandt, Strassberg (DE)

(21) Appl. No.: 12/609,513

(22) Filed: Oct. 30, 2009

(51) Int. Cl. A61F 2/84; A61F 2/82

(52) U.S. Cl. 623/1.11; 623/1.3; 623/1.15

(57) ABSTRACT

An interluminal medical treatment device configured to treat a targeted treatment site within a body lumen is provided. The interluminal medical treatment device can include an elongated generally tubular outer member. The outer member can include an expandable distal portion, an expandable proximal portion, and a central portion of limited expandability. The interluminal medical treatment device can include at least one membrane. The outer member and at least one membrane may define a housing area positioned between the proximal portion and distal portion of the outer member. The outer member and at least one membrane can be configured to redirect fluid flow within a body lumen away from the housing area inner member or inner passageway.

### BACKGROUND

1. Technical Field

The present disclosure relates to medical treatment. In particular, the present disclosure relates to systems and methods for interluminal medical treatment devices.

2. Related Technology

Stents, grafts, endoprostheses and a variety of other implantable medical devices are used in interventional procedures, such as for lining or repairing vessel walls, for filtering fluid flow, and for expanding or scaffolding occluded or collapsed vessels. Such endoprostheses can be delivered and used in an accessible body lumen of a human or animal, and can be deployed by any of a variety of recognized means.

One recognized use of implantable medical devices, such as stents, is for the treatment of atherosclerotic stenosis in blood vessels. For example, after a patient undergoes a percutaneous transluminal coronary angioplasty or similar interventional procedure, an endoprosthesis, such as a stent, can be deployed at the treatment site to improve the results of the medical procedure and to reduce the likelihood of restenosis.

However, current implantable medical devices may be limited with respect to the quantity, variation, and duration of beneficial agent treatments that the implantable medical devices can deliver to an interluminal treatment site. In addition, current endoprostheses may be unable to deliver beneficial agent in a precise, controlled, and accurate manner. Furthermore, current methods of interluminal medical treatment may be unable to isolate and protect beneficial agents from fluid flow within a lumen that can carry the beneficial agents away from the targeted treatment site.

### BRIEF SUMMARY

Embodiments of the invention relate to systems, methods, and apparatuses for interluminal medical treatment. For example, the present disclosure includes an interluminal medical treatment device configured to treat a targeted treatment site within a lumen. In particular, the device can include an elongated generally tubular outer member having an expandable proximal portion, an expandable distal portion, and a central portion having a lesser expandability than the proximal portion and distal portion. The device can also include at least one membrane coupled to the outer member. The outer member and at least one membrane may at least partially define a housing area extending around the central portion of the outer member and positioned longitudinally between the proximal portion and distal portion of the outer member. The device may also include an elongated generally tubular inner member disposed within the outer member and at least one membrane. The inner member can include at least one lumen.

It is to be understood that the foregoing general description and the following detailed description are exemplary and explanatory only and are not to be viewed as being restrictive of the present disclosure, as claimed. Further advantages of this disclosure will be apparent after a review of the following detailed description of the disclosed embodiments, which are illustrated schematically in the accompanying drawings and in the appended claims.

### BRIEF DESCRIPTION OF THE DRAWINGS

In order to describe the manner in which the above-recited and other advantages and features of the present disclosure can be obtained, a more particular description of the implementations briefly described above will be rendered by reference to specific configurations thereof which are illustrated in the appended drawings. Understanding that these drawings depict only typical configurations of the present disclosure and are not therefore to be considered to be limiting of its scope, the implementations of the present disclosure will be described and explained with additional specificity and detail through the use of the accompanying drawings.

FIG. 1A illustrates a perspective view of an example interluminal medical treatment device in accordance with an implementation of the present disclosure;

FIG. 1B illustrates a side view of the example interluminal medical treatment device of FIG. 1A;

FIG. 1C. illustrates a cut-away view of the example interluminal medical treatment device of FIG. 1B along the line 1C-1C;

FIG. 2A illustrates a perspective view of an additional example interluminal medical treatment device in accordance with an implementation of the present disclosure;

FIG. 2B illustrates a side view of the example interluminal medical treatment device of FIG. 2A;

FIG. 2C illustrates a cut-away view of the example interluminal medical treatment device of FIG. 2B along the line 2C-2C;

FIG. 3A illustrates a perspective view of a further example interluminal medical treatment device in accordance with an implementation of the present disclosure;

FIG. 3B illustrates a side view of the example interluminal medical treatment device of FIG. 3A;

FIG. 3C illustrates a cut-away view of the example interluminal medical treatment device of FIG. 3B along the line 3C-3C;

FIG. 4A illustrates a perspective view of a still further example interluminal medical treatment device in accordance with an implementation of the present disclosure;

FIG. 4B illustrates a side view of the example interluminal medical treatment device of FIG. 4A;

FIG. 4C illustrates a cut-away view of the example interluminal medical treatment device of FIG. 4B along the line 4C-4C;

FIG. 4D illustrates a cut-away view of the example interluminal medical treatment device of FIG. 4B along the line 4D-4D;

FIG. 5 illustrates an exemplary subject for an interluminal medical treatment device; and

FIGS. 6A-6F illustrate various steps in the deployment of an example interluminal medical treatment device.

It should be noted that the figures are not drawn to scale and that elements of similar structures or functions are generally represented by like reference numerals for illustrative purposes throughout the figures. It also should be noted that the figures are only intended to facilitate the description of example configurations of the present disclosure.

### DETAILED DESCRIPTION

The present disclosure relates to an interluminal medical treatment device, delivery system, and methods of interluminal medical treatment. Embodiments of the invention allow an agent to be delivered to a targeted treatment site while protecting the agent from external influence, such as fluid flow. When the interluminal medical device is deployed within a lumen proximate a targeted treatment site, the interluminal medical treatment device can define, and direct lumen fluid flow away from, a housing area. A drug or other beneficial agent can thus be introduced into the housing area and protected from fluid flow so as to be efficiently delivered to the targeted treatment site.

The interluminal medial treatment device, includes a proximal portion and a distal portion separated by a central portion. The proximal portion and the distal portion expand during deployment to engage with a lumen. The central portion, which is connected or integrally formed between the proximal portion and the distal portion, has less expandability. As a result, a housing area is formed between at least the central portion and the vessel. The proximal portion and the distal portion essentially seal or close the housing area as they engage with the walls of the vessel.

A membrane that is attached to the central portion and/or at least a portion of the distal portion and the proximal portion can prevent fluid in the lumen from entering the housing area. As described herein, however, the membrane may have some porosity. Embodiments of the invention further contemplate the delivery of a beneficial agent to the housing area. The beneficial agent is thus constrained within the housing area and can be targeted to a specific site without interference from fluid flow in the lumen.

In addition, the interluminal medical device may further include additional lumens that are longitudinally placed inside of the device. These lumens are used for various purposes such as for a guide wire during deployment and/or placement of the device, to assist in fluid flow through the device, and for delivery of a beneficial agent to the housing area.

Examples of potential beneficial agents that may be used include, but are not limited to, pharmacologic agents, anti-platelet agents, anti-inflammatory agents, anti-thrombotic agents, thrombolytic agents, and/or immunosuppressant agents.

The pharmacologic agents that can be effective in preventing restenosis can be classified into the categories of anti-proliferative agents, anti-platelet agents, anti-inflammatory agents, anti-thrombotic agents, and thrombolytic agents. Anti-proliferative agents may include, for example, crystalline rapamycin. These classes can be further sub-divided. For example, anti-proliferative agents can be anti-mitotic. Anti-mitotic agents inhibit or affect cell division, whereby processes normally involved in cell division do not take place. One sub-class of anti-mitotic agents includes vinca alkaloids. Representative examples of vinca alkaloids include, but are not limited to, vincristine, paclitaxel, etoposide, nocodazole, indirubin, and anthracycline derivatives, such as, for example, daunorubicin, daunomycin, and plicamycin. Other sub-classes of anti-mitotic agents include anti-mitotic alkylating agents, such as, for example, tauromustine, bofumustine, and fotemustine, and anti-mitotic metabolites, such as, for example, methotrexate, fluorouracil, 5-bromodeoxyuridine, 6-azacytidine, and cytarabine. Anti-mitotic alkylating agents affect cell division by covalently modifying DNA, RNA, or proteins, thereby inhibiting DNA replication, RNA transcription, RNA translation, protein synthesis, or combinations of the foregoing.

Anti-platelet agents are therapeutic entities that act by (1) inhibiting adhesion of platelets to a surface, typically a thrombogenic surface, (2) inhibiting aggregation of platelets, (3) inhibiting activation of platelets, or (4) combinations of the foregoing. Activation of platelets is a process whereby platelets are converted from a quiescent, resting state to one in which platelets undergo a number of morphologic changes induced by contact with a thrombogenic surface. These changes include changes in the shape of the platelets, accompanied by the formation of pseudopods, binding to membrane receptors, and secretion of small molecules and proteins, such as, for example, ADP and platelet factor 4. Anti-platelet agents that act as inhibitors of adhesion of platelets include, but are not limited to, eptifibatide, tirofiban, RGD (Arg-Gly-Asp)-based peptides that inhibit binding to gpIIbIIIa or αvβ3, antibodies that block binding to gpIIa-IIIb or αvβ3, anti-P-selectin antibodies, anti-E-selectin antibodies, compounds that block P-selectin or E-selectin binding to their respective ligands, saratin, and anti-von Willebrand factor antibodies. Agents that inhibit ADP-mediated platelet aggregation include, but are not limited to, disagregin and cilostazol.

Anti-inflammatory agents can also be used. Examples of these include, but are not limited to, prednisone, dexamethasone, hydrocortisone, estradiol, fluticasone, clobetasol, and non-steroidal anti-inflammatories, such as, for example, acetaminophen, ibuprofen, naproxen, and sulindac. Other examples of these agents include those that inhibit binding of cytokines or chemokines to the cognate receptors to inhibit pro-inflammatory signals transduced by the cytokines or the chemokines. Representative examples of these agents include, but are not limited to, anti-IL1, anti-IL2, anti-IL3, anti-IL 4, anti-IL8, anti-IL15, anti-IL18, anti-GM-CSF, and anti-TNF antibodies.

Anti-thrombotic agents include chemical and biological entities that can intervene at any stage in the coagulation pathway. Examples of specific entities include, but are not limited to, small molecules that inhibit the activity of factor Xa. In addition, heparinoid-type agents that can inhibit both FXa and thrombin, either directly or indirectly, such as, for example, heparin, heparin sulfate, low molecular weight heparins, such as, for example, the compound having the trademark Clivarin®, and synthetic oligosaccharides, such as, for example, the compound having the trademark Arixtra®. Also included are direct thrombin inhibitors, such as, for example, melagatran, ximelagatran, argatroban, inogatran, and peptidomimetics of binding site of the Phe-Pro-Arg fibrinogen substrate for thrombin. Another class of anti-thrombotic agents that can be delivered is factor VII/VIIa inhibitors, such as, for example, anti-factor VII/VIIa antibodies, rNAPc2, and tissue factor pathway inhibitor (TFPI).

Thrombolytic agents, which may be defined as agents that help degrade thrombi (clots), can also be used as adjunctive agents, because the action of lysing a clot helps to disperse platelets trapped within the fibrin matrix of a thrombus. Representative examples of thrombolytic agents include, but are not limited to, urokinase or recombinant urokinase, pro-urokinase or recombinant pro-urokinase, tissue plasminogen activator or its recombinant form, and streptokinase.

One or more immunosuppressant agents may be used. Immunosuppressant agents may include, but are not limited to, IMURAN® azathioprine sodium, brequinar sodium, SPANIDIN® gusperimus trihydrochloride (also known as deoxyspergualin), mizoribine (also known as bredinin), CELLCEPT® mycophenolate mofetil, NEORAL® Cylosporin A (also marketed as different formulation of Cyclosporin A under the trademark SANDIMMUNE®), PROGRAM tacrolimus (also known as FK-506), sirolimus and RAPAMUNE®, leflunomide (also known as HWA-486), glucocorticoids, such as prednisolone and its derivatives, antibody therapies such as orthoclone (OKT3) and Zenapax®, and antithymyocyte globulins, such as thymoglobulins. In addition, a crystalline rapamycin analog, A-94507, SDZ RAD (a.k.a. Everolimus), and/or other immunosuppressants.

The beneficial agents may also include stem cells, tumor treating seeds, coagulants, anticoagulants, microspheres, radioactive materials, and/or other similar agents. In a yet further configuration, the beneficial agents may include a resin that may be activated or cured by an activator, such as ultraviolet light, to form a solidified substance.

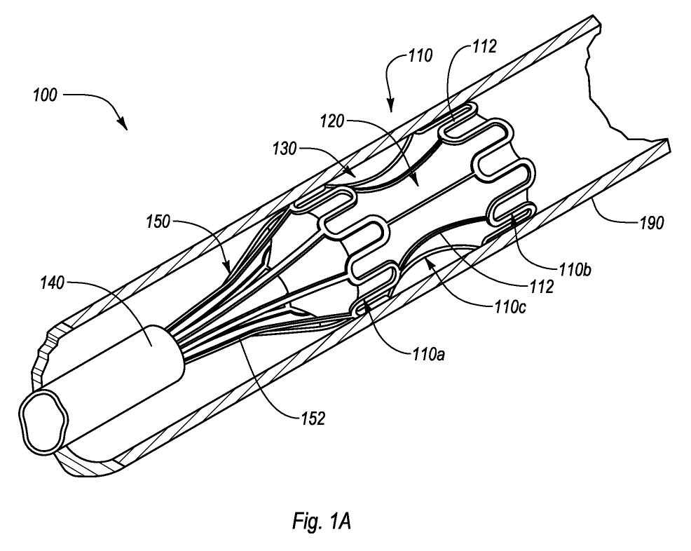

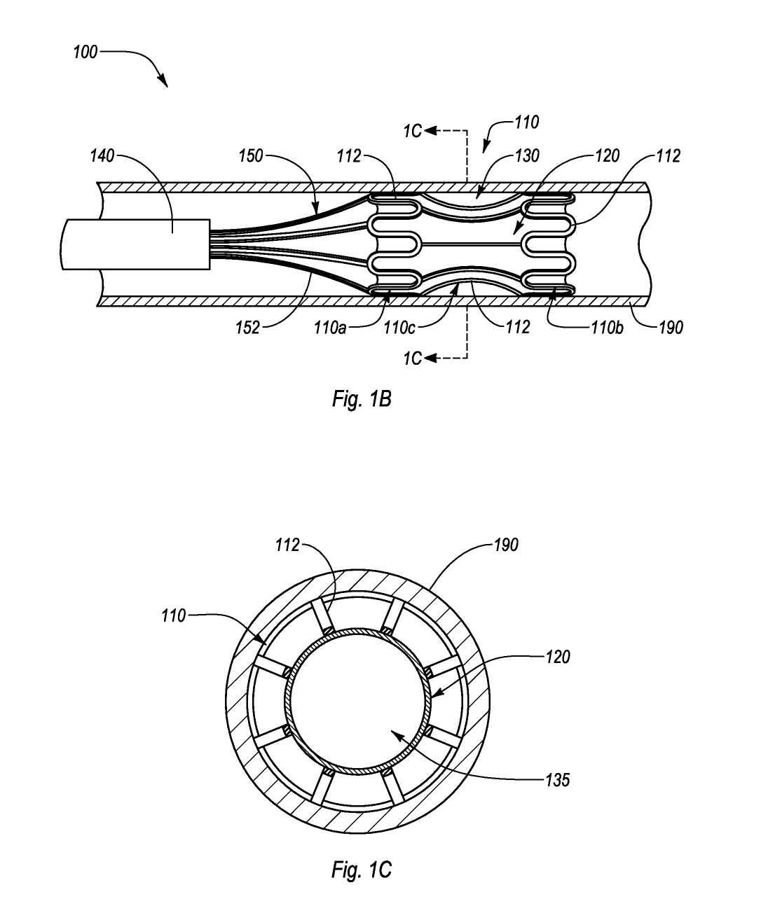

Reference is now made to FIGS. 1A-1C, which illustrate an example interluminal medical treatment device 100 deployed within a body lumen 190 in accordance with an implementation of the present disclosure. Specifically, FIG. 1A illustrates a perspective view of the interluminal medical treatment device 100 in a deployed position within the body lumen 190; FIG. 1B illustrates a side view of the interluminal medical treatment device 100 of FIG. 1A; and FIG. 1C illustrates a cut away view of the interluminal medical treatment device 100 of FIG. 1B along the line 1C-1C.

As disclosed in FIGS. 1A-1C, the interluminal medical treatment device 100 may include an outer member 110 having an expandable proximal portion 110a, an expandable distal portion 110b, and a central portion 110c of reduced expandability. In other words, the central portion 110c may have a smaller expanded outside diameter than the expanded outside diameters of the expandable proximal portion 110a and expandable distal portion 110b. The interluminal medical treatment device may also have a membrane 120 coupled to the outer member 110.

When in an expanded position, the outer member 110 and membrane 120 may define a housing area 130, into which one or more beneficial agents can be introduced. In addition, the interluminal medical treatment device 100 may include a retrieval member 150 coupled to the proximal portion 110a of the outer member 110. The interluminal medical treatment device 100 may be retrieved and/or deployed using a delivery device 140, such as a catheter.

In an embodiment, the interluminal medical treatment device 100 may be disposed within the delivery device 140 in a collapsed position. The delivery device 140 may then deliver the interluminal medical treatment device 100 near a targeted treatment site within a body lumen 190. The retrieval member 150 can then be used to deploy the interluminal medical treatment device 100. In particular, pushing the retrieval member 150 in a distal direction can force the interluminal medical treatment device 100 out of the delivery device 140. Upon leaving the delivery device 140, the outer member 110 and membrane 120 may unfold and/or expand into a deployed position, thereby engaging the inner wall of the body lumen 190 and defining a housing area 130 proximate the targeted treatment site. Beneficial agent(s) may then be introduced into the housing area 130 to treat the targeted treatment site.

In one implementation, beneficial agent(s) may be introduced into the housing area 130 while the interluminal medical treatment device 100 is being deployed from its delivery device 140. For example, an injection device, such as an injection catheter or needle, can introduce beneficial agent(s) into the housing area 130 once the distal portion 110b is deployed and while the proximal portion 110a remains in a collapsed position, such as within the delivery device 140. In a further implementation, injections of beneficial agent(s) into the housing area 130 may be completed after full deployment of the interluminal medical treatment device 100.

Once the interluminal medical treatment device 100 is deployed, the membrane 120 may redirect fluid within the body lumen 190 away from the housing area 130 and through an inner passageway 135 defined by the inside surface of the membrane 120 and/or outer member 110, thereby at least partially isolating the housing area 130 from fluid flow within the body lumen 190. As a result, beneficial agent(s) within the housing area 130 may be at least partially protected from being carried away from the targeted treatment site by fluid flow within the body lumen 190. Accordingly, treatment of a targeted treatment site within a body lumen 190 may be protected and improved.

Once treatment is completed, the interluminal medical treatment device 100 may be removed from the body lumen 190. For example, by pulling the retrieval member 150 in a proximal direction relative to the delivery device 140, the interluminal medical treatment device 100 may be collapsed and pulled into the delivery device 140. Thereafter, the interluminal medical treatment device 100 may be completely retrieved from the body.

The outer member 110 of the interluminal medical treatment device 100 may include an expandable implantable medical device or endoprosthesis, such as a stent, filter, or other endoprosthesis. The outer member 110 may be self-expanding or balloon expandable. As mentioned, the outer member 110 may include an expandable proximal portion 110a, an expandable distal portion 110b, and a central portion 110c of relatively lower expandability as compared to the proximal portion 110a and distal portion 110b. As shown in FIGS. 1A-1C, the outer member 110 may be formed using a plurality of struts 112. In particular, the proximal portion 110a and distal portion 110b may each include an expandable ring of struts 112. For example, the struts 112 of the proximal portion 110a and distal portion 110b may form a ring having a wave pattern. The central portion may include a plurality of elongated, c-shaped, and/or semi-circular struts 112 connecting the proximal portion 110a and the distal portion 110b.

In further implementations, the number and configuration of the struts may vary as desired. For example, the proximal portion 110a or distal portion 110b may each include a plurality of expandable rings or similar structures. Furthermore, the central portion 110c may include one or more rings having a lower expandability than the rings of the proximal portion 110a and distal portion 110b. In a yet further embodiment, the central portion 110c may include a plurality of v-shaped struts that angle inward toward the middle of the central portion 110c. In an even further embodiment, the outer member 110 can be formed using any expandable, generally-tubular body having a central portion 110c with a smaller expanded outside diameter than the proximal portion 110a and distal portion 110b.

The outer member 110 may be formed from various materials including shape memory materials, such as nickel titanium and/or alloys thereof, copper-aluminum-nickel and/or alloys thereof, copper chromium and/or alloys thereof, as well as stainless steel, other metals, polymers, biodegradable materials, bioresorbable materials, bioabsorbable materials, other similar materials, and/or combinations thereof. In addition, one or more beneficial agents or radio-opaque markers may be incorporated into the material of and/or coated over at least a portion of the outer member 110.

The proximal portion 110a and distal portion 110b of the example outer member 110 of FIGS. 1A-1C may have a generally tubular or cylindrical shape. However, in further configurations, the proximal portion 110a and distal portions 110b of the outer member 110 may incorporate any expanded shape capable of engaging an inner wall of the body lumen 190. For example, the proximal portion 110a or distal portion 110b may have a spherical shape, conical shape, diamond shape, spheroidal shape, and/or other similar shapes.

As mentioned, the example interluminal medical treatment device 100 of FIGS. 1A-1C may also include a membrane 120. The membrane 120 may be disposed within and coupled to an inner surface of the outer member 110. In further configurations, the membrane 120 may be disposed outside and coupled to an outer surface of the outer member 110.

The example membrane 120 may extend along the central portion 110c of the outer member 110 and at least partially along the proximal portion 110a and distal portion 110b. In further configurations, however, the membrane 120 may extend further or lesser along the portions 110a, 110b, 110c of the outer member 110. In addition, although the example membrane 120 of FIGS. 1A-1C is a single piece, in still further configurations, the membrane 120 may comprise a plurality of pieces coupled to the outer member 110.

The membrane 120 can incorporate any porosity desired for a particular configuration. For example, the membrane 120 can be substantially permeable, semi-permeable, impermeable, substantially porous, or non-porous. The porosity or permeability of the membrane 120 may be adjusted as desired depending on the type of beneficial agent to be administered and the desired treatment. For example, a porous or permeable material may be used for the membrane 120 if a residual flow through the membrane 120 is desired. Furthermore, the porosity or permeability of the membrane 120 may vary from one portion of the membrane 120 to another.

The membrane 120 may comprise any of a number of different materials. For example, the membrane 120 may include polymers, such as plastics, polyurethanes, synthetic fabrics, elastomers, and/or other similar materials. In one example embodiment, the membrane 120 may comprise a polymer film. In additional embodiments, the membrane 120 may comprise Polyamide (e.g.: Nylon, Pebax), PE, PET, Silicone (polysiloxane), PTFE, e-PFTE, other similar materials, or combinations thereof. In further embodiments, the membrane 120 may include multiple layers of different polymers combined to form the membrane 120. In yet further embodiments, the membrane 120 may comprise blends or compounds of different polymers.

The membrane 120 may be manufactured from the various polymers in different structures, such as non-woven (fleece), woven (fabric), spinning (e-spinning or mechanical spinning), foam, and/or foil. In additional embodiments, the membrane 120 may be engineered from biologic material such as skin or endothelium derived from humans or animals. In further embodiments, the membrane 120 may also consist of metals and alloys thereof, such as stainless steel, cobalt-chromium, Nickel-Titanium (Nitinol), similar metals/alloys, or combinations thereof. In yet further embodiments, metals/alloys may be structured into the membrane 120 such as woven, spun, fabric, and/or thin film (e.g. Nitinol thin film) metals/alloys. In a further embodiment, the membrane 120 may comprise one or more balloons, such as balloons configured to expand the outer member 110 and/or drug eluting balloons.

In a yet further embodiment, the membrane 120 may be integral with the outer member 110. For example, the membrane 120 and outer member 110 may be of a unitary construct. In particular, the membrane 120 may be formed with and be an integral part of the outer member 110.

As introduced, the outer member 110 and/or membrane 120 may define a housing area 130 extending annularly around the interluminal medical treatment device 100. In one implementation, the housing area 130 may be located proximate the central portion 110c and between the proximal portion 110a and distal portion 110b. As a result, when the interluminal medical treatment device 100 is deployed within a body lumen 190, as shown in FIGS. 1A-1C, the outer member 110 and membrane 120 may redirect fluid flow within the body lumen 190 away from the housing area 130 and through an inner passageway 135 defined by the inside surface of the membrane 120. Accordingly, the interluminal medical treatment device 100 may at least partially isolate and protect the contents of the housing area 130, while maintaining fluid flow within the body lumen 190. In additional configurations, the outer member 110 and membrane 120 may define multiple housing areas 130 or the housing area 130 may extend around only a portion of the body lumen 190. In a yet further configuration, the outer member 110 and membrane 120 may be deployed so as to contain a targeted treatment site within the housing area 130 without exerting physical forces directly onto the targeted treatment site.

As mentioned above, the interluminal medical treatment device 100 may also include a retrieval member 150 configured to retrieve and/or deploy the interluminal medical treatment device 100. As shown in FIGS. 1A-1C, the retrieval member 150 may be coupled to the outer member 110, such as to the proximal portion 110a. The retrieval member 150 may also be configured to at least partially extend into the delivery device 140. For example, the retrieval member 150 may include a plurality of elongated struts 152 coupled to the proximal portion 110a and configured to extend at least partially into the delivery device 140. In further configurations, the retrieval member 150 may include additional structural elements to assist in retrieving and/or deploying the interluminal medical treatment device 100, such as a hook or other similar elements.

Pushing the retrieval member 150 in a distal direction relative to the delivery device 140 may force the interluminal medical treatment device 100 out of the delivery device 140 and into a deployed position. Alternatively, pulling the retrieval member 150 in a proximal direction relative to the delivery device 140 can force the interluminal medical treatment device 100 into the delivery device 140 and into a collapsed position.

As introduced above, once the interluminal medical treatment device 100 is deployed or is in the process of being deployed proximate a targeted treatment site, one or more beneficial agents may be introduced into the housing area 130. Because the inner surface of the body lumen 190 defines an exterior boundary to the housing area 130, beneficial agent(s) contained within the housing area 130 may be maintained against the surface being treated. The membrane 120 and/or outer member 110 may also protect the beneficial agent(s) from fluid flow within the body lumen 190 that would otherwise dilute the beneficial agent(s) or carry the beneficial agent(s) away from the targeted treatment site. As a result, treatment of a targeted site within the body lumen 190 may be accomplished in a more precise, controlled, and efficient manner.

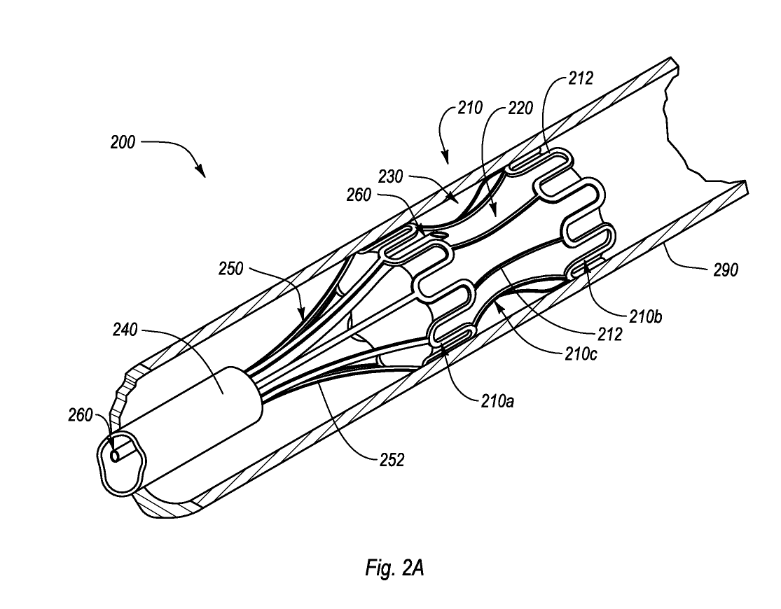

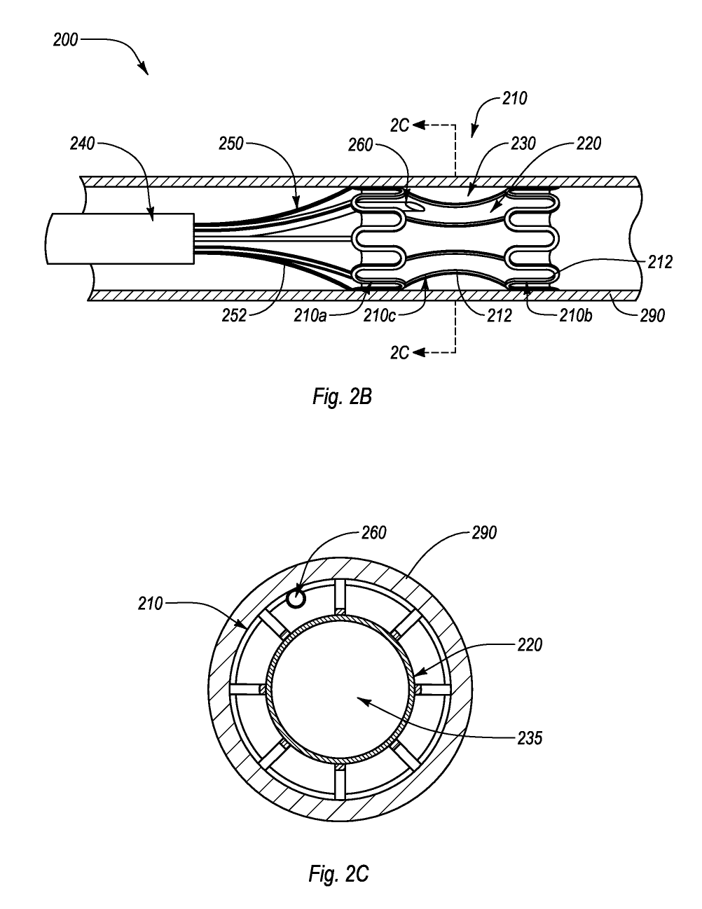

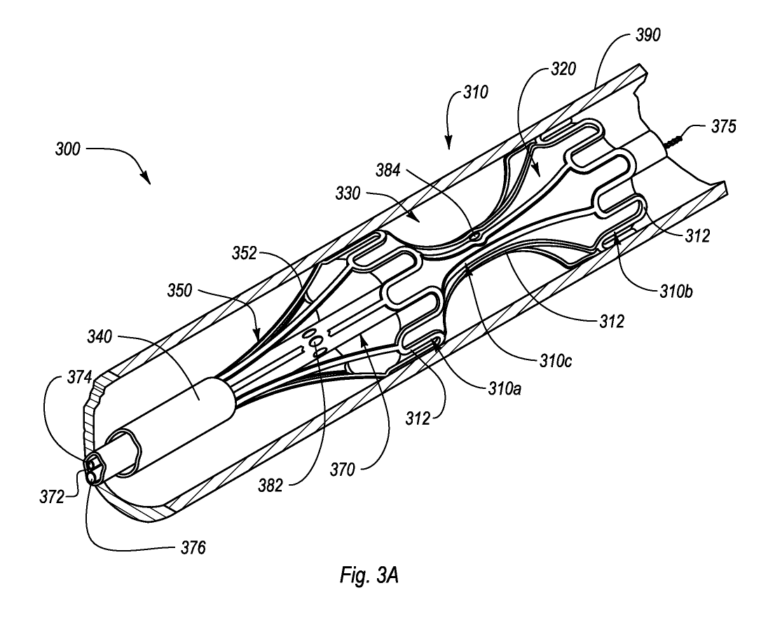

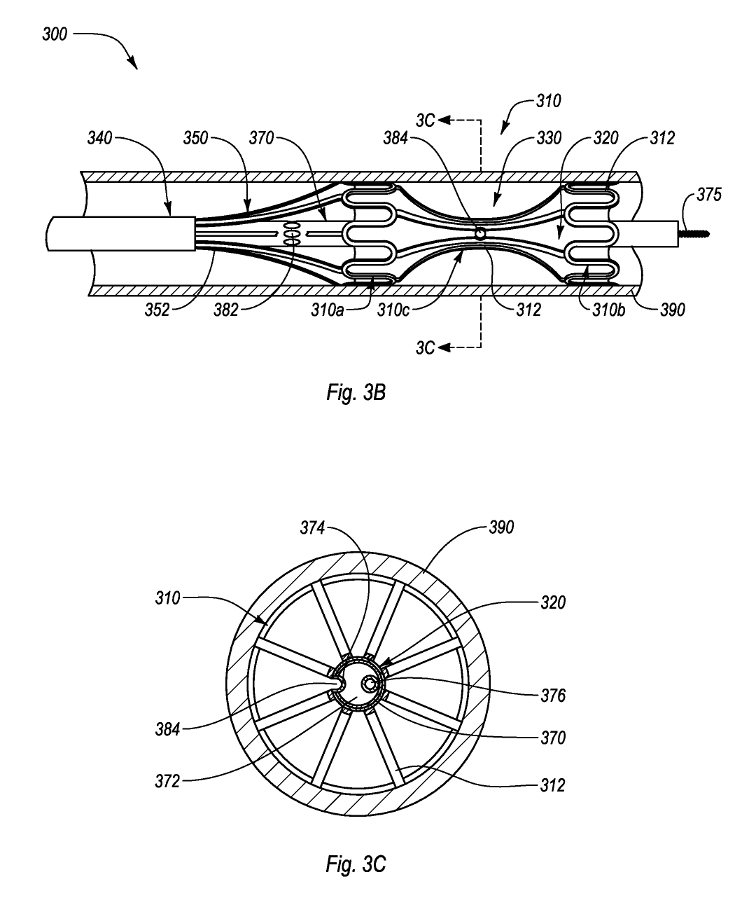

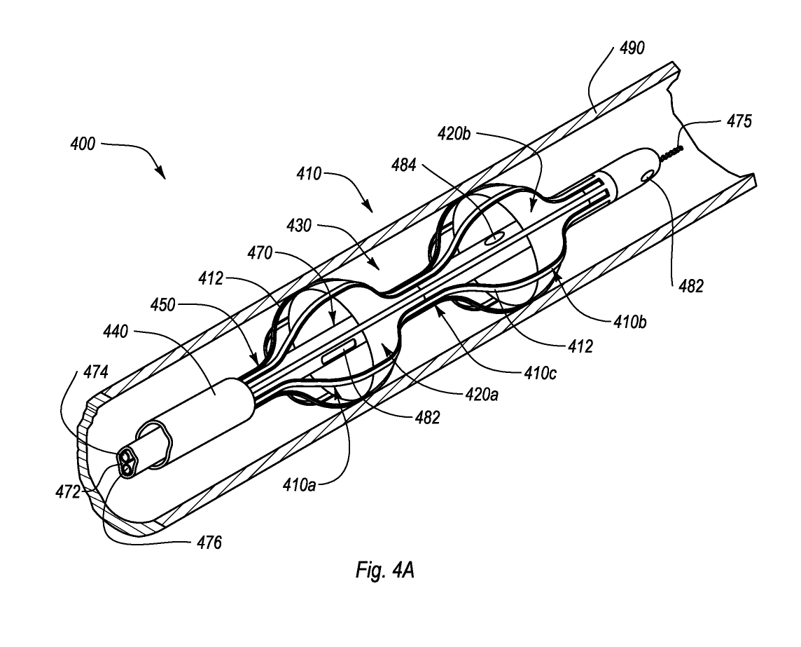

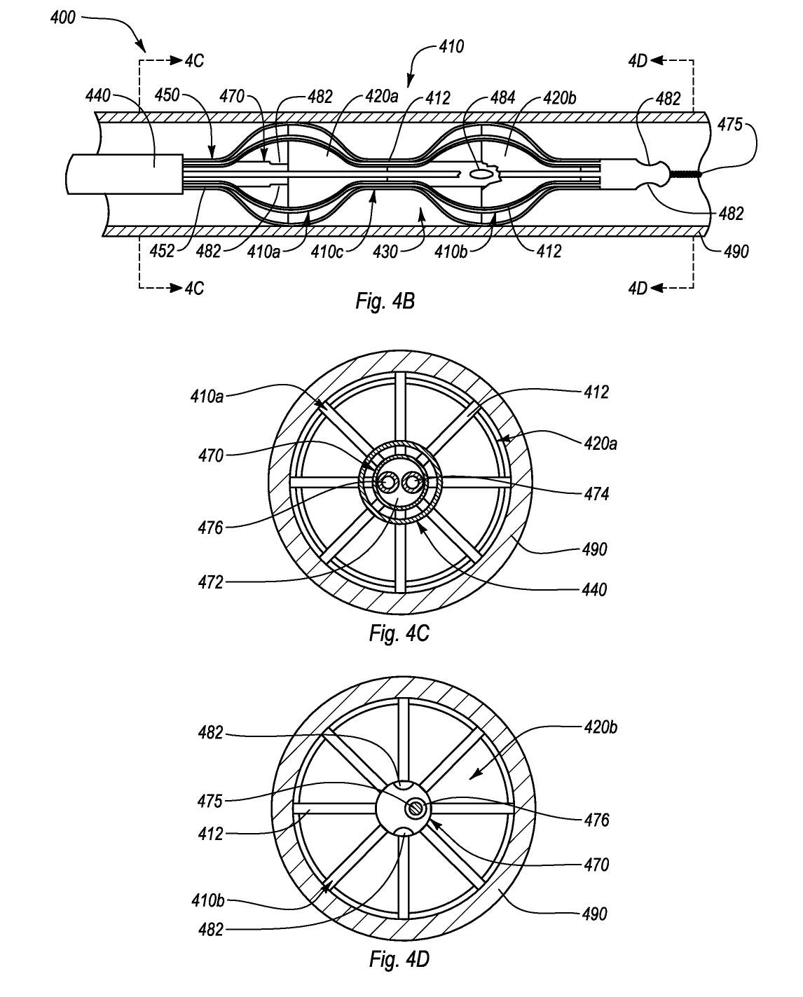

The interluminal medical treatment device 100 may incorporate at least one component of the interluminal medical treatment devices 200, 300, 400, 500, and 600 described in connection with FIGS. 2-6, respectively. The following non-limiting list of examples indicates the interchangeability of at least some of the components of the interluminal medical treatment devices 100, 200, 300, 400, 500, and 600 described herein. For instance, the interluminal medical treatment device 100 may include one or more injection devices (e.g., 260, as shown in FIGS. 2A-2C). In another example, the interluminal medical treatment device 100 may further incorporate an inner member (e.g., 370 and 470, as shown in FIGS. 3 and 4, respectively) disposed within the outer member 110 and membrane 120.

Reference is now made to FIGS. 2A-2C, which disclose another example interluminal medical treatment device 200 in accordance with an implementation of the present disclosure. Specifically, FIG. 2A illustrates a perspective view of the interluminal medical treatment device 200 in a deployed position within a body lumen 290; FIG. 2B illustrates a side view of the interluminal medical treatment device 200 of FIG. 2A; and FIG. 2C illustrates a cut away view of the interluminal medical treatment device 200 of FIG. 2B along the line 2C-2C.

The example interluminal medical treatment device 200 of this configuration may be functionally similar to the example interluminal medical treatment device 100 previously described above and shown in FIGS. 1A-1C in most respects, wherein certain features will not be described in relation to this configuration wherein those components may function in the manner as described above and are hereby incorporated into this additional configuration described below. Like structures and/or components are given like reference numerals. Additionally, the interluminal medical treatment device 200 may incorporate at least one component of the interluminal medical treatment devices 100, 300, 400, 500, and 600 described in connection with FIGS. 1, and 3-6, respectively.

As disclosed in FIGS. 2A-2C, the interluminal medical treatment device 200 may include an outer member 210 having an expandable proximal portion 210a, an expandable distal portion 210b, and a central portion 210c of reduced expandability. The interluminal medical treatment device may also have a membrane 220 coupled to the outer member 210. When in an expanded position, the outer member 210 and membrane 220 may define a housing area 230, into which one or more beneficial agents may be introduced. The interluminal medical treatment device 200 may also include an injection device 260, such as a needle and/or catheter, disposed through the delivery device 240 and extending at least partially into the housing area 230. In addition, the interluminal medical treatment device 200 may include a retrieval member 250 coupled to the outer member 210. The interluminal medical treatment device 200 may be retrieved and/or deployed using a delivery device 240, such as a catheter.

In one embodiment, the interluminal medical treatment device 200 may be disposed within the delivery device 240 in a collapsed position. The delivery device 240 may then deliver the interluminal medical treatment device 200 near a targeted treatment site within a body lumen 290. The retrieval member 250 may then be used to deploy the interluminal medical treatment device 200. In particular, pushing the retrieval member 250 in a distal direction may move the interluminal medical treatment device 200 out of the delivery device 240. Upon leaving the delivery device 240, the outer member 210 and membrane 220 may unfold and/or expand into a deployed position, thereby engaging the inner wall of the body lumen 290 and defining a housing area 230 proximate a targeted treatment site. Using the injection device 260, beneficial agent(s) may then be introduced into the housing area 230 to treat the targeted site.

Once the interluminal medical treatment device 200 is deployed, the membrane 220 and/or outer member 210 may redirect fluid within the body lumen 290 away from the housing area 230 and through an inner passageway 235 defined by the inside surface of the membrane 220 and outer member 210, thereby at least partially isolating the housing area 230 from fluid flow within the body lumen 290. As a result, beneficial agent(s) within the housing area 230 may be protected from being diluted or carried away from the targeted treatment site by fluid flow within the body lumen 290.

Once treatment is completed, the interluminal medical treatment device 200 may be removed from the body lumen 290. For example, by pulling the retrieval member 250 in a proximal direction relative to the delivery device 240, the interluminal medical treatment device 200 may be collapsed and pulled into the delivery device 240 and thereafter, completely retrieved from the body.

The outer member 210 of the interluminal medical treatment device 200 may include an expandable implantable medical device or prosthesis, such as a stent, filter, or other endoprosthesis. The outer member 210 may include an expandable proximal portion 210a, an expandable distal portion 210b, and a central portion 210c of reduced expandability. In one example configuration, the outer member 210 may be formed using a plurality of struts 212. In particular, the proximal portion 210a and distal portion 210b may each include an expandable ring of struts 212. The central portion may include a plurality of elongated struts 212 connecting the proximal portion 210a and the distal portion 210b. In particular, the elongated struts 212 of the central portion 210c may taper and/or curve radially inward from the proximal portion 210a and distal portion 210b to the middle of the central portion 210c. As a result, the deployed position of the central portion 210c may have a smaller diameter than the proximal portion 210a or distal portion 210b.

In further implementations, the number and configuration of the struts may vary as desired. For example, the proximal portion 210a or distal portion 210b may each include a plurality of expandable rings or similar structures. Furthermore, the central portion 210c can include one or more rings having a lesser expandability than the rings of the proximal portion 210a and distal portion 210b. In a further embodiment, the outer member 210 can be formed using any expandable, generally-tubular body.

The outer member 210 may be formed from various materials including shape memory materials, such as nickel titanium and/or alloys thereof, copper-aluminum-nickel and/or alloys thereof, copper chromium and/or alloys thereof, as well as stainless steel, other metals, polymers, biodegradable materials, bioresorbable materials, bioabsorbable materials, other similar materials, and/or combinations thereof. In addition, one or more beneficial agents may be incorporated into the material of and/or coated over at least a portion of the outer member 210.

The proximal portion 210a and distal portion 210b of the example outer member 210 of FIGS. 2A-2C may have a generally tubular or cylindrical shape. However, in further configurations, the proximal portion 210a and distal portion 210b of the outer member 210 may incorporate any expanded shape capable of engaging an inner wall of the body lumen 290. For example, the proximal portion 210a or distal portion 210b may have a spherical shape, conical shape, diamond shape, spheroidal shape, and/or other similar shapes.

As mentioned, the example interluminal medical treatment device 200 of FIGS. 2A-2C also includes a membrane 220. The example membrane 220 can be disposed within and coupled to an inner surface of the outer member 210. In further configurations, the membrane 220 may be disposed outside and coupled to an outer surface of the outer member 210.

The membrane 220 can extend along the central portion 210c of the outer member 210 and at least partially along the proximal portion 210a and distal portion 210b. In a further embodiment, the membrane 220 may extend along substantially the entire length of the outer member 210, as shown in the example illustrated in FIGS. 2A-2C. In further configurations, the membrane 220 may comprise multiple pieces coupled to the outer member 210.

The membrane 220 can incorporate any porosity and/or permeability desired for a particular configuration. For example, the membrane 220 can be porous, semi-porous, impermeable, or other similar configurations. As a result, the membrane 220 can at least partially redirect fluid flow within a body lumen 290 when the interluminal medical treatment device 200 is in a deployed position.

The outer member 210 and/or membrane 220 can define a housing area 230 extending annularly around the interluminal medical treatment device 200 proximate the central portion 210c. As a result, when the interluminal medical treatment device 200 is deployed within a body lumen 290, as shown in FIGS. 2A-2C, the outer member 210 and membrane 220 can redirect fluid flow within the body lumen 290 away from the housing area 230 and through an inner passageway 235 defined by the inside surface of the membrane 220. As a result, the interluminal medical treatment device 200 can at least partially isolate and protect the contents of the housing area 230, while maintaining fluid flow within the body lumen 290. In additional configurations, the outer member 210 and membrane 220 may define multiple housing areas 230 or the housing area 230 may extend around only a portion of the interluminal medical treatment device 200.

As mentioned above, the interluminal medical treatment device 200 may also include a retrieval member 250 configured to retrieve and/or deploy the interluminal medical treatment device 200. For example, as shown in FIGS. 2A-2C, the retrieval member 250 can be coupled to the outer member 210, such as to the proximal portion 210a. The retrieval member 250 can also be configured to at least partially extend into the delivery device 240. In one configuration, the retrieval member 250 can include a plurality of elongated struts 252 coupled to the proximal portion 210a and configured to extend at least partially into the delivery device 240. In further configurations, the retrieval member 250 may include additional structural elements, such as a hook or other similar elements for retrieving and/or deploying the interluminal medical treatment device 200.

Pushing the retrieval member 250 in a distal direction relative to the delivery device 240 can force the interluminal medical treatment device 200 out of the delivery device 240 and into a deployed position. Alternatively, pulling the retrieval member 250 in a proximal direction relative to the delivery device 240 can force the interluminal medical treatment device 200 into the delivery device 240 and into a collapsed position. Accordingly, the retrieval member 250 may assist in the deployment and retrieval of the interluminal medical treatment device 200.

The interluminal medical treatment device 200 can also include an injection device 260, such as a needle and/or catheter, to introduce beneficial agent(s) into the housing area 230. In the example configuration of FIGS. 2A-2C, the injection device 260 passes through the delivery device 240 and extends at least partially into the housing area 230. The injection device 260 may extend from a point external to a patient receiving treatment through the lumen 290 and into the housing area 230. As a result, a physician may use a syringe or other external device to introduce a beneficial agent into the housing area 230 through the injection device 260. In one example embodiment, the injection device 260 can be coupled to an automated device, whether internal or external to the patient, configured to automatically introduce a predetermined amount of beneficial agent into the housing area 230 at predetermined time intervals.

In one example configuration, the injection device 260 may be temporarily positioned within the housing area 230 and then removed once a beneficial agent is introduced into the housing area 230. In a further configuration, the injection device 260 may remain within the housing area 230 for an extended period of time, thereby facilitating sustained and/or ongoing treatments of a targeted treatment site within the body lumen 290. In a yet further configuration, the injection device 260 may be connected to the outer member 210, membrane 220, and/or retrieval member 250. In additional configurations, the interluminal medical treatment device 200 may include multiple injection devices 260 extending into the housing area 230, through which the same or different beneficial agents may be introduced. Furthermore, one or more beneficial agents may be introduced through a single injection device 260.

The size of the injection device 260 may vary as necessary for a particular configuration. In one example embodiment, the injection device 260 may include a 27 gauge, or larger, needle. The injection device 260 may also be configured for the delivery of cells to the housing area 230.

Once the interluminal medical treatment device 200 is deployed proximate a targeted treatment site, one or more beneficial agents may be introduced into the housing area 230. Because the inner surface of the body lumen 290 may define an exterior boundary to the housing area 230, beneficial agent(s) contained within the housing area 230 can be maintained directly against the inner wall of the body lumen 290 being treated. The outer member 210 and membrane 220 can also protect the beneficial agent(s) from fluid flow within the body lumen 290 that would otherwise carry the beneficial agent(s) away from the targeted treatment site. Accordingly, treatment of a targeted site within the body lumen 290 can be accomplished in a more precise, controlled, and efficient manner.

Further aspects of the present disclosure are disclosed in FIGS. 3A-3C, which illustrate yet another example interluminal medical treatment device 300 in accordance with an implementation of the present disclosure. Specifically, FIG. 3A illustrates a perspective view of the interluminal medical treatment device 300 in a deployed position within a body lumen 390; FIG. 3B illustrates a side view of the interluminal medical treatment device 300 of FIG. 3A; and FIG. 3C illustrates a cut away view of the interluminal medical treatment device 300 of FIG. 3B along the line 3C-3C. The example interluminal medical treatment device 300 of this configuration may be functionally similar to the example interluminal medical treatment devices 100 and 200 previously described above and shown in FIGS. 1 and 2 in most respects, wherein certain features will not be described in relation to this configuration wherein those components may function in the manner as described above and are hereby incorporated into this additional configuration described below. Like structures and/or components are given like reference numerals. Additionally, the interluminal medical treatment device 300 may incorporate at least one component of the interluminal medical treatment devices 100, 200, 400, 500, and 600 described in connection with FIGS. 1, 2, and 4-6, respectively.

As disclosed in FIGS. 3A-3C, the example interluminal medical treatment device 300 includes an outer member 310 having an expandable proximal portion 310a, an expandable distal portion 310b, and a central portion 310c of reduced expandability. In other words, the central portion 310c may have a smaller expanded outside diameter than the expanded outside diameters of the expandable proximal portion 310a and expandable distal portion 310b. The interluminal medical treatment device may also have a membrane 320 coupled to the outer member 310.

When in an expanded position, the outer member 310 and membrane 320 may define a housing area 330, into which one or more beneficial agents may be introduced. The interluminal medical treatment device 300 may also include a generally elongated and/or tubular inner member 370 disposed within the outer member 310 and membrane 320. The inner member 370 may include a beneficial agent lumen 374, a guidewire lumen 376, and/or a perfusion lumen 372. The inner member 370 may also include one or more corresponding perfusion ports 382 and/or beneficial agent ports 384. In addition, the interluminal medical treatment device 300 can include a retrieval member 350 coupled to the proximal portion 310a of the outer member 310. Finally, the interluminal medical treatment device 300 can be retrieved and/or deployed using a delivery device 340, such as a catheter.

In one embodiment, the interluminal medical treatment device 300 can be disposed within the delivery device 340 in a collapsed position. The delivery device 340 can then deliver the interluminal medical treatment device 300 near a targeted treatment site within a body lumen 390. The retrieval member 350 can then be used to deploy the interluminal medical treatment device 300. In particular, pushing the retrieval member 350 in a distal direction can force the interluminal medical treatment device 300 out of the delivery device 340. Upon leaving the delivery device 340, the outer member 310 and membrane 320 can unfold and/or expand into a deployed position, thereby engaging the inner wall of the body lumen 390 and defining a housing area 330 proximate a targeted treatment site. Beneficial agent(s) can then be introduced into the housing area 330 via the beneficial agent lumen 374 and corresponding beneficial agent port 384 to treat the targeted treatment site.

Once the interluminal medical treatment device 300 is deployed, the membrane 320 can redirect fluid within the body lumen 390 away from the housing area 330 and through the perfusion lumen 374 of the inner member 370, thereby at least partially isolating the housing area 330 from fluid flow within the body lumen 390. As a result, beneficial agent(s) within the housing area 330 can be protected from being carried away from the targeted treatment site by fluid flow within the body lumen 390.

Once treatment is completed, the interluminal medical treatment device 300 can be removed from the body lumen 390. For example, by pulling the retrieval member 350 in a proximal direction relative to the delivery device 340, the interluminal medical treatment device 300 can be collapsed and pulled into the delivery device 340. Thereafter, the interluminal medical treatment device 300 can be completely retrieved from the body.

As mentioned, the interluminal medical treatment device 300 can include a generally tubular outer member 310, such as a stent or other endoprosthesis. The outer member 310 can include an expandable proximal portion 310a, an expandable distal portion 310b, and a central portion 310c of reduced expandability. In the example configuration of FIGS. 3A-3C, the outer member 310 may be formed using a plurality of struts 312. In particular, the proximal portion 310a and distal portion 310b may each include an expandable ring of struts 312. The central portion can include a plurality of elongated struts 312 connecting the proximal portion 310a and the distal portion 310b. In particular, the elongated struts 312 of the central portion 310c can taper and/or curve radially inward from the proximal portion 310a and distal portion 310b to the longitudinal middle of the central portion 310c. In a further configuration, the central portion 310c may be at least partially coupled to the inner member 370. As a result, the deployed position of the central portion 310c has a smaller diameter than the deployed position of the proximal portion 310a and distal portion 310b.

The outer member 310 may be formed from various materials including nickel titanium and/or alloys thereof, copper-aluminum-nickel and/or alloys thereof, copper chromium and/or alloys thereof, as well as stainless steel, polymers, biodegradable materials, bioresorbable materials, bioabsorbable materials, other similar materials, and/or combinations thereof. In addition, one or more beneficial agents may be incorporated into the material of and/or coated over at least a portion of the outer member 310.

The proximal portion 310a and distal portion 310b of the example outer member 310 of FIGS. 3A-3C can have a generally tubular or cylindrical shape. However, in further configurations, the proximal portion 310a and distal portion 310b of the outer member 310 may incorporate any expanded shape capable of engaging an inner wall of the body lumen 390. For example, the proximal portion 310a or distal portion 310b may have a spherical shape, conical shape, diamond shape, spheroidal shape, and/or other similar shapes.

As mentioned, the example interluminal medical treatment device 300 of FIGS. 3A-3C also includes a membrane 320. The membrane 320 may be disposed within and coupled to an inner surface of the outer member 310. In further configurations, the membrane 320 may be disposed outside and coupled to an outer surface of the outer member 310.

The membrane 320 can extend along the central portion 310c of the outer member 310 and at least partially along the proximal portion 310a and distal portion 310b. In a further configuration, the example membrane 320 may extend along substantially the entire length of the outer member 310, as shown in the example disclosed in FIGS. 3A-3C. In addition, the membrane 320 can comprise a plurality of pieces coupled to the outer member 310.

The membrane 320 can incorporate any porosity and/or permeability desired for a particular configuration. For example, the membrane 320 can be porous, semi-porous, impermeable, or other similar configurations. As a result, the membrane 320 can at least partially redirect fluid flow within a body lumen 390 when the interluminal medical treatment device 300 is in a deployed position.

The outer member 310 and/or membrane 320 can define a housing area 330 extending annularly around the interluminal medical treatment device 300 about the central portion 310c and longitudinally between the proximal portion 310a and distal portion 310b. As a result, when the interluminal medical treatment device 300 is deployed within a body lumen 390, as shown in FIGS. 3A-3C, the outer member 310 and membrane 320 can redirect fluid flow within the body lumen 390 away from the housing area 330 and through a perfusion lumen 372 of the inner member 370. As a result, the interluminal medical treatment device 300 can at least partially isolate and protect the contents of the housing area 330, while maintaining fluid flow within the body lumen 390. In additional configurations, the outer member 310 and membrane 320 may define multiple housing areas 330 or the housing area 330 may extend around only a portion of the interluminal medical treatment device 300.

As mentioned above, the interluminal medical treatment device 300 may also include a retrieval member 350 configured to retrieve and/or deploy the interluminal medical treatment device 300. For example, as shown in FIGS. 3A-3C, the retrieval member 350 can be coupled to the outer member 310, such as to the proximal portion 310a. The retrieval member 350 can also be configured to at least partially extend into the delivery device 340. In one configuration, the retrieval member 350 can include a plurality of elongated struts 352 coupled to the proximal portion 310a and configured to extend at least partially into the delivery device 340. In further configurations, the retrieval member 350 may include additional structural elements, such as a hook or other similar elements.

Pushing the retrieval member 350 in a distal direction relative to the delivery device 340 can force the interluminal medical treatment device 300 out of the delivery device 340 and into a deployed position. Alternatively, pulling the retrieval member 350 in a proximal direction relative to the delivery device 340 can force the interluminal medical treatment device 300 into the delivery device 340 and into a collapsed position. Accordingly, the retrieval member 350 may assist in the deployment and retrieval of the interluminal medical treatment device 300.

As introduced above, the interluminal medical treatment device 300 can also include an inner member 370 extending longitudinally along the length of the interluminal medical treatment device 300 and being disposed within the membrane 320 and/or outer member 310. In particular, the inner member 370 can be coupled to an inner surface of the membrane 320 and/or outer member 310. The inner member 370 may comprise any of a number of different materials such as metals, plastics, and/or similar materials. Furthermore, the materials used to form the inner member 370 may be flexible or rigid.

The inner member 370 may define and/or contain one or more lumens. For example, the inner member 370 may include a perfusion lumen 372, a beneficial agent lumen 374, and/or a guidewire lumen 376. As shown in the example configuration, the beneficial agent lumen 374 and/or guidewire lumen 376 can be disposed within the perfusion lumen 372.

The inner member 370 may include one or more perfusion ports 382 in fluid communication with the perfusion lumen 372. For example, the inner member may include one or more perfusion ports 382 proximal of the housing area 330 to allow fluid flow within the body lumen 390 that has been redirected by the membrane 320 and/or outer member 310 to enter into and travel through the perfusion lumen 372 of the inner member 370. The inner member 370 may also include one or more perfusion ports 382 distal of the housing area 330 to allow fluid flow to exit the perfusion lumen 372. As a result, the housing area 330 can be isolated and protected as fluid flow within the body lumen 390 is redirected through the inner member 370.

The inner member 370 can include one or more beneficial agent ports 384 in fluid communication with the beneficial agent lumen 374 and extending into the housing area 330. Once the interluminal medical treatment device 300 is deployed, one or more beneficial agents can be introduced into the housing area 330 via the beneficial agent lumen 374 and corresponding beneficial agent port(s) 384. As a result, the beneficial agent(s) can be used to treat a targeted treatment site within the body lumen 390. In a further configuration, the inner member 370 may include a plurality of beneficial agent lumens 374 with corresponding beneficial agent ports 384 extending into the housing area 330 for the delivery of a plurality of distinct beneficial agents into the housing area 330.

The beneficial agent lumen 374 may extend from a point external to a patient receiving treatment through the lumen 390 and into the housing area 330. As a result, a physician may use a syringe or other external device to introduce a beneficial agent into the housing area 330 through the beneficial agent lumen 374. In one example embodiment, the injection device 360 can be coupled to an automated device, whether internal or external to the patient, configured to automatically inject a predetermined amount of beneficial agent into the housing area 330 at predetermined time intervals.

The inner member 370 may include a guidewire lumen 376 extending longitudinally therein. A corresponding guidewire 375 may pass through the guidewire lumen 376 and be used to position the interluminal medical treatment device 300 proximate a targeted treatment site within the body lumen 390. Once the interluminal medical treatment device 300 is positioned and/or deployed, the guidewire 375 may be removed from or remain within the guidewire lumen 376.

Reference is now made to FIGS. 4A-4C, which disclose a further example interluminal medical treatment device 400 in accordance with an implementation of the present disclosure. Specifically, FIG. 4A illustrates a perspective view of the interluminal medical treatment device 400 in a deployed position within a body lumen 490; FIG. 4B illustrates a side view of the interluminal medical treatment device 400 of FIG. 4A; FIG. 4C illustrates a cut away view of the interluminal medical treatment device 400 of FIG. 4B along the line 4C-4C; and FIG. 4D illustrates a cut away view of the interluminal medical treatment device 400 of FIG. 4B along the line 4D-4D.

The example interluminal medical treatment device 400 of this configuration may be functionally similar to the example interluminal medical treatment devices 100, 200, and 300 previously described above and shown in FIGS. 1-3 in most respects, wherein certain features will not be described in relation to this configuration wherein those components may function in the manner as described above and are hereby incorporated into this additional configuration described below. Like structures and/or components are given like reference numerals. Additionally, the interluminal medical treatment device 400 may incorporate at least one component of the interluminal medical treatment devices 100, 200, 300, 500, and 600 described in connection with FIGS. 1-3 and 5-6, respectively.

As disclosed in FIGS. 4A-4C, the example interluminal medical treatment device 400 may include an outer member 410 having an expandable proximal portion 410a, an expandable distal portion 410b, and a central portion 410c of reduced expandability. In other words, the central portion 410c may have a smaller expanded outside diameter than the expanded outside diameters of the expandable proximal portion 410a and expandable distal portion 410b. The interluminal medical treatment device may also include a proximal membrane 420a disposed within the proximal portion 410a and a distal membrane 420b disposed within the distal portion 410b. When in an expanded position, the outer member 410 and membranes 420a, 420b may define a housing area 430, into which one or more beneficial agents can be introduced. The interluminal medical treatment device 400 can include an inner member 470 disposed within the outer member 410 and/or membranes 420a, 420b. The inner member 470 can include a beneficial agent lumen 474, a guidewire lumen 476, and/or a perfusion lumen 472. The inner member 470 may also include one or more corresponding perfusion ports 482 and/or beneficial agent ports 484. In addition, the interluminal medical treatment device 400 can include a retrieval member 450 coupled to the proximal portion 410a of the outer member 410. Finally, the interluminal medical treatment device 400 can be retrieved and/or deployed using a delivery device 440, such as a catheter.

In a configuration, the interluminal medical treatment device 400 can be disposed within the delivery device 440 in a collapsed position. The delivery device 440 can then deliver the interluminal medical treatment device 400 near a targeted treatment site within a body lumen 490. The retrieval member 450 can then be used to deploy the interluminal medical treatment device 400. In particular, pushing the retrieval member 450 in a distal direction can force the interluminal medical treatment device 400 out of the delivery device 440.

Upon leaving the delivery device 440, the outer member 410 and membranes 420a, 420b can unfold and/or expand into a deployed position, thereby engaging the inner wall of the body lumen 490 and defining a housing area 430 proximate a targeted treatment site. Beneficial agent(s) can then be introduced into the housing area 430 via the beneficial agent lumen 474 and corresponding beneficial agent port 484 to treat the targeted treatment site.

Once the interluminal medical treatment device 400 is deployed, the membranes 420a, 420b can redirect fluid within the body lumen 490 away from the housing area 430 and through the perfusion lumen 474 of the inner member 470, thereby at least partially isolating and protecting the housing area 430 from fluid flow within the body lumen 490. As a result, beneficial agent(s) within the housing area 430 can be protected from being diluted and/or carried away from the targeted treatment site by fluid flow within the body lumen 490.

Once treatment is completed, the interluminal medical treatment device 400 can be removed from the body lumen 490. For example, by pulling the retrieval member 450 in a proximal direction relative to the delivery device 440, the interluminal medical treatment device 400 can be collapsed and pulled into the delivery device 440. Thereafter, the interluminal medical treatment device 400 can be completely retrieved from the body.

The outer member 410 of the interluminal medical treatment device 400, may include may include an expandable endoprosthesis, such as a stent, filter, or other endoprosthesis. The outer member 410 can include an expandable proximal portion 410a, an expandable distal portion 410b, and a central portion 410c of reduced expandability. In one example configuration, the outer member 410 may be formed using a plurality of struts 412. In particular, the example outer member 410 may formed using a plurality of elongated struts 412 that extend longitudinally from the proximal portion 410a to the distal portion 410b. The struts 412 may curve, taper, and/or bend to form the proximal portion 410a and distal portion 410b with greater expanded diameters than the central portion 410c. For example, the struts may be c-shaped, v-shaped, rectangularly shaped, other similar shapes, and/or combinations of the same shapes, to provide the desired dimensions to the proximal portion 410a, distal portion 410b, and central portion 410c. In one example configuration, the elongated struts 412 may be radially spaced apart and coupled together only at the ends of the interluminal medical treatment device 400.

The outer member 410 may be formed from various materials including nickel titanium and/or alloys thereof, copper-aluminum-nickel and/or alloys thereof, copper chromium and/or alloys thereof, stainless steel, polymers, biodegradable materials, bioresorbable materials, bioabsorbable materials, other similar materials, and/or combinations thereof. In addition, one or more beneficial agents may be incorporated into the material of and/or coated over at least a portion of the outer member 410.

The proximal portion 410a and distal portion 410b of the example outer member 410 of FIGS. 4A-4C can have a generally spherical expanded shape. However, in further configurations, the proximal portion 410a and distal portions 410b of the outer member 410 may incorporate any expanded shape capable of engaging an inner wall of the body lumen 490. For example, the proximal portion 410a or distal portion 410b may have a cylindrical shape, conical shape, diamond shape, spheroidal shape, and/or other similar shapes.

As mentioned, the example interluminal medical treatment device 400 may include membranes 420a, 420b coupled to the outer member 410 and/or inner member 470. In particular, the proximal membrane 420a can be coupled to the proximal portion 410a of outer member 410. In addition, the distal membrane 420b may be coupled to the distal portion 410b of outer member 410.

The membranes 420a, 420b can be coupled to the inner member 470 and extend at least partially along the proximal portion 410a and distal portion 410b, respectively. In particular, the membranes 420a, 420b may each be umbrella-shaped extending approximately half way into the proximal and distal portions 410a, 410b, respectively. In further configurations, the membranes 420a, 420b may extend along substantially the entire length of the proximal portion 410a and distal portion 410b, respectively. Although a plurality of membranes 420a, 420b are disclosed, in a further implementation, the interluminal medical treatment device 400 may include a single membrane that extends along the proximal portion 410a, distal portion 410b, and/or central portion 410c.

The membranes 420a, 420b can incorporate any porosity and/or permeability desired for a particular configuration. For example, the membranes 420a, 420b can be porous, semi-porous, impermeable, or other similar configurations. In addition, the proximal membrane 420a can have a greater or lesser permeability and/or porosity than the distal membrane 420b. The porosity or permeability of the membrane 420 may be adjusted as desired depending on the type of beneficial agent to be administered and the desired treatment. For example, a porous or permeable material may be used for the membrane 420 if a residual flow through the membrane 420 is desired. Furthermore, the porosity or permeability of the membrane 420 may vary from one portion of the membrane 420 to another.

The outer member 410 and/or membranes 420a, 420b can define a housing area 430 extending annularly around the interluminal medical treatment device 400 about the central portion 410c and longitudinally between the proximal portion 410a and distal portion 410b. As a result, when the interluminal medical treatment device 400 is deployed within a body lumen 490, as shown in FIGS. 4A-4C, the outer member 410 and membranes 420a, 420b can redirect fluid flow within the body lumen 490 away from the housing area 430 and through a perfusion lumen 472 of the inner member 470. As a result, the interluminal medical treatment device 400 can at least partially isolate and protect the contents of the housing area 430, while maintaining fluid flow within the body lumen 490. In additional configurations, the outer member 410 and membranes 420a, 420b may define multiple housing areas 430 or the housing area 430 may extend around only a portion of the interluminal medical treatment device 400.

As mentioned above, the interluminal medical treatment device 400 may include a retrieval member 450 configured to retrieve and/or deploy the interluminal medical treatment device 400. For example, as shown in FIGS. 4A-4C, the retrieval member 450 can be coupled to the outer member 410, such as to the proximal portion 410a. The retrieval member 450 can also be configured to at least partially extend into the delivery device 440. In one configuration, the retrieval member 450 can include a plurality of elongated struts 452 coupled to the proximal portion 410a and configured to extend at least partially into the delivery device 440. In a further configuration, the struts 452 of the retrieval member 450 can be extensions of the struts 412 of the outer member 410. In further configurations, the retrieval member 450 may include additional structural elements, such as a hook or other similar elements.

Pushing the retrieval member 450 in a distal direction relative to the delivery device 440 can force the interluminal medical treatment device 400 out of the delivery device 440 and into a deployed position. Alternatively, pulling the retrieval member 450 in a proximal direction relative to the delivery device 440 can force the interluminal medical treatment device 400 into the delivery device 440 and into a collapsed position. Accordingly, the retrieval member 450 may assist in the deployment and retrieval of the interluminal medical treatment device 400.

As introduced above, the interluminal medical treatment device 400 can include an inner member 470 extending longitudinally along the length of the interluminal medical treatment device 400 and being disposed within the membranes 420a, 420b and/or outer member 410. In particular, the inner member 470 can be coupled to an inner surface of the membranes 420a, 420b and/or outer member 410. The inner member 470 may comprise any of a number of different materials such as metals, plastics, and/or similar materials, and/or combinations thereof, including any materials that may be used for the outer member 410. Furthermore, the materials used to form the inner member 470 may be flexible or rigid.

The inner member 470 may define and/or contain one or more lumens. For example, the inner member 470 may include a perfusion lumen 472, a beneficial agent lumen 474, and a guidewire lumen 476. As shown in the example configuration, the beneficial agent lumen 474 and/or guidewire lumen 476 can be disposed within the perfusion lumen 472.

The inner member 470 may include one or more perfusion ports 482 in fluid communication with the perfusion lumen 472. For example, the inner member may include one or more perfusion ports 482 proximal of the housing area 430 to allow fluid flow within the body lumen 490 that has been redirected by the membranes 420a, 420b and/or outer member 410 to enter into and travel through the perfusion lumen 472 of the inner member 470. The inner member 470 may also include one or more perfusion ports 482 distal of the housing area 430 to allow fluid flow to exit the perfusion lumen 472. As a result, the housing area 430 can be isolated and protected as fluid flow within the body lumen 490 is maintained and redirected through the inner member 470.

The inner member 470 can include one or more beneficial agent ports 484 in fluid communication with the beneficial agent lumen 474 and extending into the housing area 430. Once the interluminal medical treatment device 400 is deployed, one or more beneficial agents can be introduced into the housing area 430 via the beneficial agent lumen 474 and corresponding port(s) 484. As a result, the beneficial agent(s) can be used to treat a targeted treatment site within the body lumen 490. In a further configuration, the inner member 470 may include a plurality of beneficial agent lumens 474 with corresponding beneficial agent ports 484 extending into the housing area 430 for the delivery of a plurality of distinct beneficial agents into the housing area 430.

The beneficial agent lumen 474 may extend from a point external to a patient receiving treatment through the lumen 490 and into the housing area 430. As a result, a physician may use a syringe or other external device to introduce a beneficial agent into the housing area 430 through the beneficial agent lumen 474. In one example embodiment, the injection device 460 can be coupled to an automated device, whether internal or external to the patient, configured to automatically inject a predetermined amount of beneficial agent into the housing area 430 at predetermined time intervals.

The inner member 470 may include a guidewire lumen 476 extending longitudinally therein. A corresponding guidewire 475 may pass through the guidewire lumen 476 and be used to position the interluminal medical treatment device 400 proximate a targeted treatment site within the body lumen 490. Once the interluminal medical treatment device 400 is positioned and/or deployed, the guidewire 475 may be removed from or remain within the guidewire lumen 476.

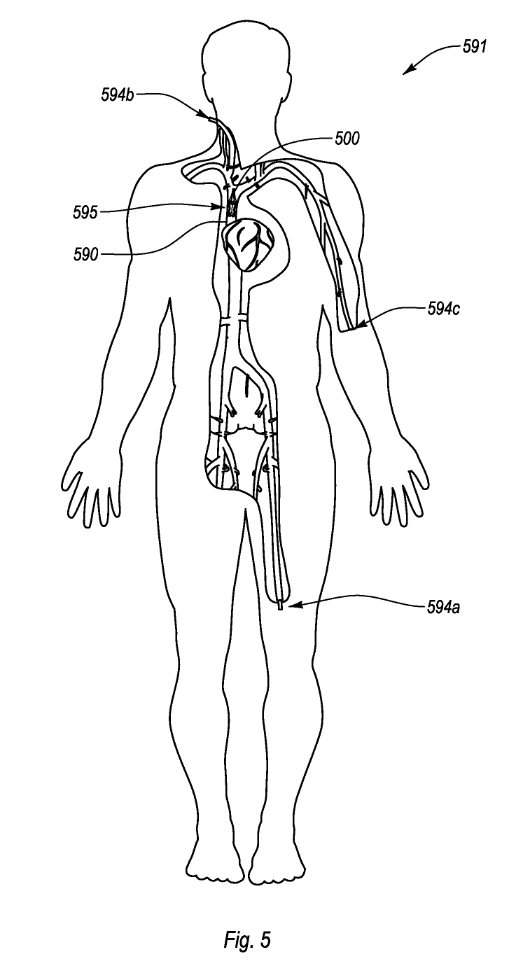

FIG. 5 illustrates an exemplary subject 591 for an interluminal medical treatment device 500. The interluminal medical treatment device 500 may be functionally similar to the interluminal medical treatment devices 100, 200, 300, and 400 previously described above and shown in FIGS. 1-4 in most respects, wherein certain features will not be described in relation to this configuration wherein those components may function in the manner as described above and are hereby incorporated into the configuration described below. Like structures and/or components are given like reference numerals. Additionally, the interluminal medical treatment device 500 may incorporate at least one component of the interluminal medical treatment devices 100, 200, 300, 400, and 600 described in connection with FIGS. 1-4 and 6, respectively.

The interluminal medical treatment device 500 may be implanted in a body lumen 590 of the subject 591. As illustrated in FIG. 5, the interluminal medical treatment device 500 may be inserted and/or retrieved through an access site 594a, 594b, 594c. In the present embodiment, the access site may include a femoral artery access site 594a, a jugular vein access site 594b, a radial vein access site 594c, femoral vein, brachial vein, brachial artery, other access sites, or combinations thereof.

For instance, the interluminal medical treatment device 500 may be inserted through the femoral artery access site 594a and retrieved through the jugular or radial vein access site 594b, 594c. In another example, the interluminal medical treatment device 500 may be inserted through the jugular vein access site 594b and retrieved through the femoral artery or radial vein access site 594a, 594c. In a further example, the interluminal medical treatment device 500 may be inserted through the radial vein access site 594c and retrieved through the femoral artery or jugular vein access site 594a, 594b.

In a yet further example, the interluminal medical treatment device 500 may be inserted and retrieved through the radial vein access site 594c. Additionally, the interluminal medical treatment device 500 may be inserted and retrieved through the jugular vein access site 594b. Further, the interluminal medical treatment device 500 may be inserted and retrieved through the femoral artery access site 594a.

The interluminal medical treatment device 500 may be deployed near a deployment site 595. In one implementation, the deployment site 595 may include a location within a coronary artery. In other implementations, other deployment sites may be used. For example, the deployment site 595 may include central and peripheral arteries and veins, vena cavas, bile ducts, esophagus, colons, trachea, large bronchi, ureters, and urethra.

Once deployed at the deployment site 595, the interluminal medical treatment device 500 may at least partially define a housing area (not shown) adjacent to the inner wall of the body lumen 590. A medical care provider can introduce a beneficial agent into the housing area 530, which may be at least partially protected from fluid flow within the body lumen 590.

In one implementation, the interluminal medical treatment device 500 may be coupled to a beneficial agent lumen (not shown). In particular, the beneficial agent lumen can be part of or coupled to an injection device (e.g., 260, FIGS. 2A-2C) or an inner member (e.g., 370, 470, FIGS. 3-4) of the interluminal medical treatment device 500. The beneficial agent lumen may extend from the deployed interluminal medical treatment device 500 to a point external to the subject 591 through one of the access sites 594a, 594b, 594c. For example, the beneficial agent lumen can extend from the deployment site 595 through the radial vein access site 594c to a point external to the subject 591. Accordingly, a physician or other medical care provider can access the beneficial agent lumen in order to introduce one or more beneficial agents into the housing area 530 through the beneficial agent lumen.

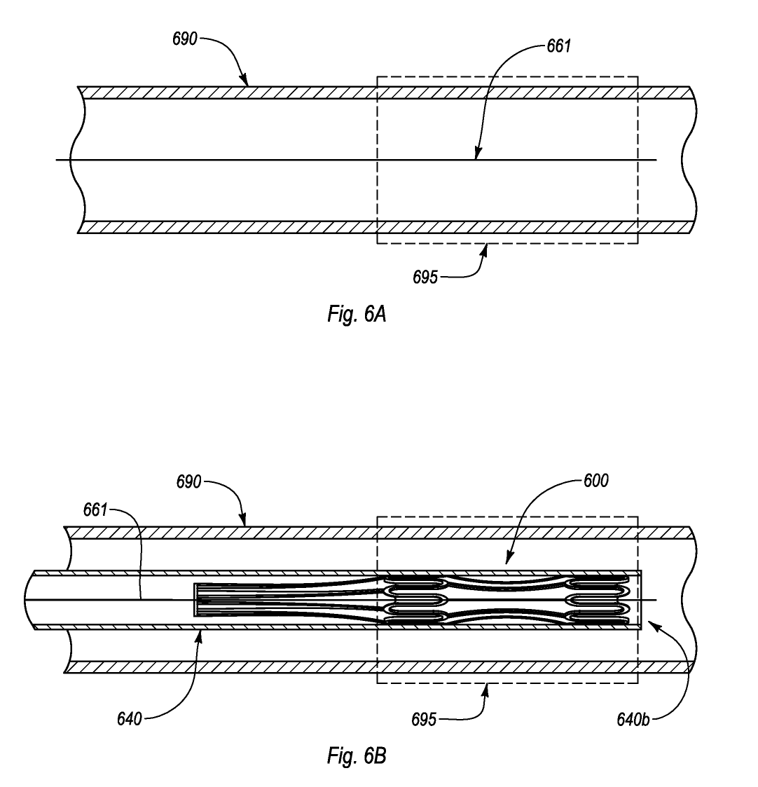

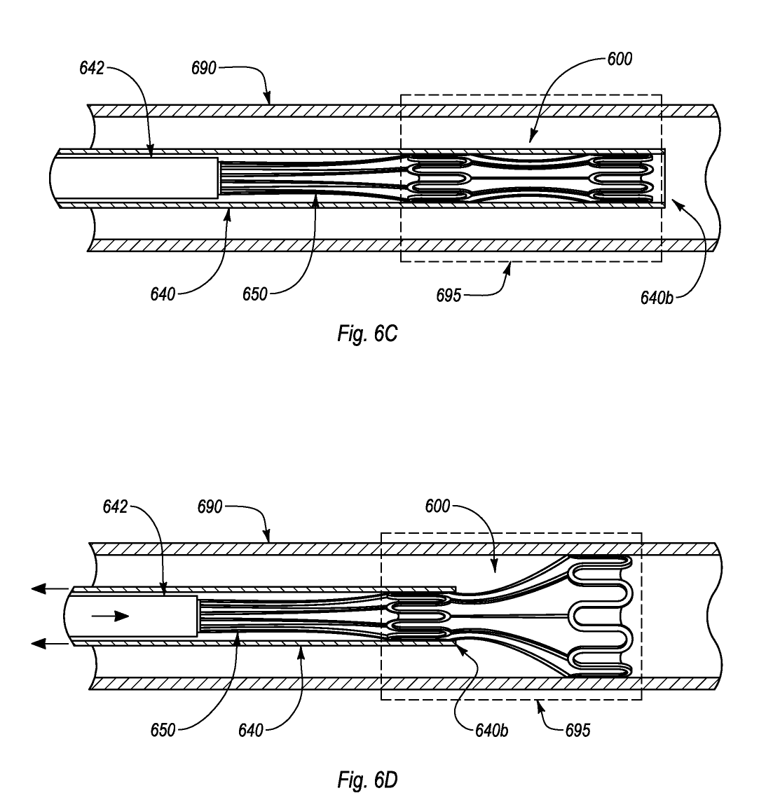

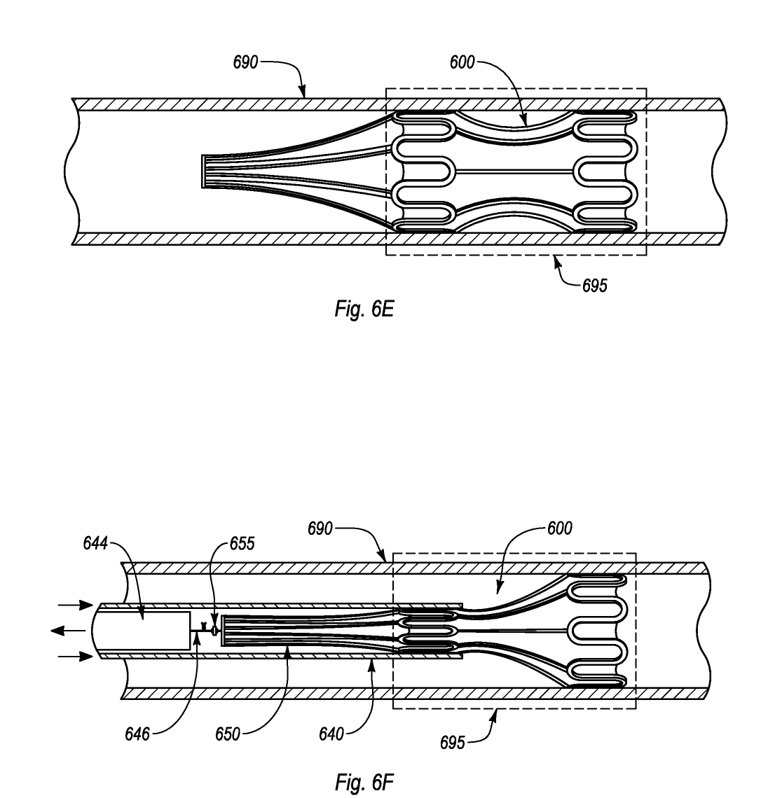

FIGS. 6A-6F illustrate various steps in the deployment of an interluminal medical treatment device 600. The interluminal medical treatment device 600 may be functionally similar to the interluminal medical treatment devices 100, 200, 300, 400, 500 previously described above and shown in FIGS. 1-5 in most respects, wherein certain features will not be described in relation to this configuration wherein those components may function in the manner as described above and are hereby incorporated into the configuration described below. Like structures and/or components are given like reference numerals. Additionally, the interluminal medical treatment device 600 may incorporate at least one component of the interluminal medical treatment devices 100, 200, 300, 400, and 500 described in connection with FIGS. 1-5, respectively.

FIG. 6A illustrates a deployment site 695 within a body lumen 690 with a guidewire 661 partially inserted therethrough. The guidewire 661 may be inserted through an access site (shown as 594a, 594b, 594c in FIG. 5) toward the deployment site 695. The guidewire 661 may be used to locate the deployment site 695. Once used to locate the deployment site 695, the guidewire 661 may either remain within the lumen 690 or may be removed. In other configurations, other methods may be used in addition to or instead of a guidewire 661. For example, an imaging device, such as a fluoroscope, x-ray, and/or other imaging device may be used to locate the deployment site 695.

As shown in FIG. 6B, a delivery device 640 may use the guidewire 661 to guide a distal end 640b of the delivery device 640 toward the deployment site 695. An interluminal medical treatment device 600 may be disposed within the delivery device 640 in a collapsed state. While in the collapsed state, the interluminal medical treatment device 600 may be radially compressed and/or longitudinally elongated with respect to a deployed state.

A deployment member 642 may be inserted through the delivery device 640, as shown in FIG. 6C. The deployment member 642 may be used to deploy the interluminal medical treatment device 600. In the configuration shown in FIG. 6D, the deployment member 642 may move the interluminal medical treatment device 600 toward the distal end 640b of the delivery device 640 while the delivery device 640 may remain generally stationary.

The deployment member 642 may move the interluminal medical treatment device 600 by abutting the retrieval member 650 of the interluminal medical treatment device 600. The deployment member 642 may include a receiving area (not shown), such as a convex portion configured and dimensioned to receive the retrieval member 650, to facilitate deploying the interluminal medical treatment device 600.

In one implementation, as shown in FIG. 6D, the delivery device 640 may be retracted while the deployment member 642 may remain generally stationary. In a further implementation, the deployment member 642 may be advanced while the delivery device 640 may remain stationary. In other configurations, the delivery device 640 and/or the deployment member 642 may cooperate to facilitate deployment of the interluminal medical treatment device 600. For instance, the delivery device 640 may be retracted while the deployment member 642 may urge the interluminal medical treatment device 600 toward the distal end 640b of the delivery device 640.

FIG. 6E illustrates a deployed interluminal medical treatment device 600 within the body lumen 690. In the deployed configuration, the implantable interluminal medical treatment device 600 may engage an inside surface of the body lumen 690. For example, the proximal portion 610a and distal portion 610b of the interluminal medical treatment device 600 may engage the inside surface of the body lumen 690. In the deployed configuration, the interluminal medical treatment device 600 may be longitudinally reduced with respect to a collapsed configuration.

In a further implementation, as shown in FIG. 6F, the interluminal medical treatment device 600 may include a retrieval mechanism 655 operatively connected to the retrieval member 650. The interluminal medical treatment device 600 may be engaged by a retrieval apparatus 644 disposed within the delivery device 640. The retrieval apparatus 644 may also include a retrieving mechanism 646, such as a hook and/or other retaining mechanism, configured to engage the retrieval mechanism 655 of the interluminal medical treatment device 600.

Upon engaging the retrieval mechanism 655, the retrieval apparatus 644 may urge the interluminal medical treatment device 600 into the delivery device 640. For example, urging the interluminal medical treatment device 600 toward the delivery device 640 may facilitate disengaging the interluminal medical treatment device 600 from the inside surface of the lumen 690.

In one implementation, the retrieval apparatus 644 may urge the interluminal medical treatment device 600 in a proximal direction while the delivery device 640 remains stationary. In a further implementation, the delivery device 640 may be advanced in a distal direction while the retrieval apparatus 644 maintains the interluminal medical treatment device 600 stationary. In a yet further configuration, the delivery device 640 and the retrieval apparatus 644 may both move in generally opposite directions to urge the interluminal medical treatment device 600 into the delivery device 640 into a compressed state.

Once the implantable device 600 is within the delivery device 640, the delivery device 640 and implantable device 600 may be withdrawn through an access site (shown as 594a, 594b, 594c in FIG. 5).

While the present disclosure has been described, disclosed, illustrated and shown in various terms of certain embodiments or modifications, the scope of the present disclosure is not intended to be, nor should it be deemed to be, limited thereby and such other modifications or embodiments as may be suggested by the teachings herein are particularly reserved especially as they fall within the breadth and scope of the claims here appended.

What is claimed is:

1. An interluminal medical treatment device configured to treat a targeted treatment site within a lumen, the device comprising:
an elongated generally tubular outer member including an expandable proximal portion, an expandable distal portion, and a central portion having a lesser expandability than the proximal portion and the distal portion;
at least one membrane coupled to the outer member, the outer member and at least one membrane at least partially defining a housing area extending around the central portion of the outer member and positioned longitudinally between the proximal portion and distal portion of the outer member; and
an elongated generally tubular inner member disposed within the outer member and at least one membrane, the inner member including at least one lumen.

2. The device of claim 1, in which the at least one membrane is coupled to an inner surface of the outer member and extends through the central portion and at least partially into the distal and proximal portions of the outer member.

3. The device of claim 1, in which the at least one membrane comprises a proximal membrane disposed at least partially within the proximal portion of the outer member and a distal membrane disposed at least partially within the distal portion of the outer member.

4. The device of claim 3, in which the proximal membrane has a different permeability or porosity than the distal membrane.

5. The device of claim 1, in which the outer member is formed of shape memory material.

6. The device of claim 1, in which the inner member is coupled to the at least one membrane.

7. The device of claim 1, in which the inner member includes a perfusion lumen.

8. The device of claim 7, in which the inner member includes at least one perfusion port in a proximal portion of the inner member and at least one perfusion port in a distal portion of the inner member, the perfusion ports being in fluid communication with the perfusion lumen.

9. The device of claim 1, in which the inner member includes a beneficial agent lumen in fluid communication with the housing area by way of a beneficial agent port.

10. The device of claim 1, in which the inner member includes a guidewire lumen.

11. The device of claim 1, further comprising a retrieval member coupled to the outer member, the retrieval member being configured to deploy the device from a delivery device and retrieve the device into the delivery device.

12. The device of claim 11, in which the retrieval member includes a plurality of elongated struts coupled at a distal end to the outer member and coupled at a proximal end to each other.

13. An interluminal medical treatment device configured to treat a targeted treatment site within a lumen, the device comprising:
an elongated generally tubular outer member including an expandable proximal portion, an expandable distal portion, and a central portion having a reduced expandability compared to the proximal portion and distal portion;
at least one membrane coupled to the outer member, the outer member and at least one membrane defining a housing area extending around the central portion of the outer member and positioned longitudinally between the proximal portion and distal portion of the outer member; and
an injection device extending at least partially into the housing area.

14. The device of claim 13, further comprising a plurality of injection devices extending at least partially into the housing area.

15. The device of claim 13, in which the proximal portion or distal portion of the outer member comprises an expandable ring of struts.

16. The device of claim 13, in which the outer member comprises a plurality of radially spaced, longitudinally extending struts coupled together at a distal end of the outer member.

17. A method for treating a targeted treatment site within a lumen comprising:
deploying an interluminal medical treatment device within the lumen proximate the targeted treatment site, the device comprising:
an elongated generally tubular outer member including an expandable proximal portion, an expandable distal portion, and a central portion having a reduced expandability compared to the proximal portion and distal portion;
at least one membrane coupled to the outer member, the outer member and at least one membrane defining a housing area extending around the central portion of the outer member and positioned longitudinally between the proximal portion and distal portion of the outer member; and
introducing at least one beneficial agent into the housing area.

18. The method according to claim 17, further comprising redirecting fluid flow within the lumen through an elongated inner member disposed within the outer member and at least one membrane.

19. The method according to claim 18, further comprising redirecting fluid flow into at least one perfusion port in a proximal portion of the inner member, through a perfusion lumen of the inner member, and out of at least one perfusion port in a distal portion of the inner member.

20. The method according to claim 17, further comprising introducing at least one beneficial agent into the housing area through an injection device extending at least partially into the housing area.

21. The method according to claim 18, further comprising introducing at least one beneficial agent into the housing area through a beneficial agent lumen disposed within the inner member, the beneficial agent lumen being in fluid communication with the housing area.

22. An interluminal medical treatment device delivery system comprising:
a tubular delivery device configured to hold the interluminal medical treatment device in a collapsed position and deliver the interluminal medical treatment device to a targeted treatment site; and
a medical treatment device disposed within the tubular delivery device, the medical treatment device comprising:
an elongated generally tubular outer member including an expandable proximal portion, an expandable distal portion, and a central portion having a reduced expandability compared to the proximal portion and distal portion;
at least one membrane coupled to the outer member, the outer member and at least one membrane defining a housing area extending around the central portion of the outer member and positioned longitudinally between the proximal portion and distal portion of the outer member; and
an elongated inner member disposed within the outer member and at least one membrane; and
a retrieval member coupled to the outer member and configured to assist in deployment of the interluminal medical treatment device from the tubular delivery device and retrieval of the interluminal medical treatment device into the tubular delivery device.

23. The delivery system of claim 22, in which the inner member includes a perfusion lumen in fluid communication with at least one perfusion port in a proximal portion of the inner member and with at least one perfusion port in a distal portion of the inner member.

24. The delivery system of claim 22, in which the inner member includes a beneficial agent lumen in fluid communication with the housing area.

25. The delivery system of claim 22, in which the at least one membrane comprises a proximal membrane disposed at least partially within the proximal portion of the outer member and a distal membrane disposed at least partially within the distal portion of the outer member.
